(19) United States
(12) Patent Application Publication
(10) Pub. No.: US 2010/0288167 A1
(43) Pub. Date: Nov. 18, 2010

(54) LOW DIOL CONTENT MONOFUNCTIONAL ALKOXYPOLYALKYLENE GLYCOLS AND PROCESS FOR PRODUCING THEM

(76) Inventors: David B. Wurm, Pearland, TX (US); Robert H. Whitmarsh, Lake Jackson, TX (US); Peirre T. Varineau, Lake Jackson, TX (US); Bruce A. Barner, Sweeny, TX (US); John G. Pendergast, JR., Lake Jackson, TX (US); Kirk R. Thompson, Lake Jackson, TX (US)

Correspondence Address: The Dow Chemical Company, P.O. BOX 1967, Midland, MI, 48641, US

(21) Appl. No.: 12/863,531

(22) PCT Filed: Jan. 7, 2009
(86) PCT No.: PCT/US09/30248

§ 371 Date: Jul. 19, 2010

Related U.S. Application Data
(60) Provisional application No. 61/025,551, filed on Feb. 1, 2008.

(51) Int. Cl. C08K 5/053; C07C 31/02; C09K 3/00

(52) U.S. Cl. 106/499; 568/840; 252/182.24; 252/182.14

(57) ABSTRACT

Provided is a process for preparing low diol content monofunctional polyalkylene glycols. The process includes introducing the initiator feed in two portions (a first and second initiator) and drying only the first initiator to remove water. The first and second initiators can be the same or different. Also provided are new monofunctional polyalkylene glycol compositions.

### CROSS-REFERENCE TO PRIOR APPLICATION

This application claims the benefit of U.S. Provisional Application No. 61/025,551 filed Feb. 1, 2008.

### FIELD OF THE INVENTION

The invention relates to monofunctional polyalkylene glycols containing low levels of diol contamination, and to processes for their preparation.

### BACKGROUND OF THE INVENTION

Monofunctional polyalkylene glycols, such as monomethoxy polyethylene glycols (MPEGs), are used in a wide variety of applications, and particularly in applications where crosslinking of the glycol additive is undesirable. For example, MPEGs are used in large quantities in the production of polyalkylene glycol-based plasticizers and dispersants in cement applications. In these applications, the MPEGs are grafted onto an unsaturated carboxylic acid backbone via esterification, forming a macromonomer. The macromonomer is further polymerized to make materials known as superplasticizers. Other important applications using MPEGs include polyurethane and polyurethane prepolymer production. MPEGs are also used in the manufacture of benzonatate, the active ingredient in some cough suppressants.

MPEGs are produced by the reaction of an alcohol with an epoxide, such as ethylene oxide:

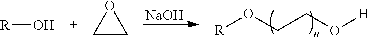

MPEG formulations prepared according to the known processes generally also contain difunctional polyethylene glycols (R═H in the above product) (referred to herein as “diols”) as a contaminant. The diols are primarily caused by the presence of water, which is generated as a byproduct of the alkoxylation of the alcohol by the hydroxide catalyst. Water is also present as the aqueous solvent of the catalyst, and may further be present in the starting raw materials. The water reacts with the epoxide to form the diols as a byproduct. Other difunctional impurities in the raw materials, such ethylene glycol, diethylene glycol, and other difunctional species, may also contribute to the presence of difunctional polyethylene glycol contaminants.

Diols in MPEG formulations act as crosslinking agents during subsequent applications. Many applications that use MPEGs, however, rely on MPEGs precisely in order to avoid crosslinking reactions. For example, in the cement applications mentioned above, diol contaminants form diesters upon esterification and lead to crosslinking and gel formation during superplasticizer production. In pharmaceutical applications, crosslinking can lead to formation of unacceptable contaminants.

In order to address the presence of diols, WO 2006/061110 describes a process in which the alcohol and base catalyst mixture (see above equation) is first dried prior to alkoxylation and polymerization. To facilitate this drying step, the reference requires that the alcohol have a higher boiling point/lower vapor pressure than water.

Several problems are associated with the process of WO 2006/061110, including that the reference's distillative drying step can result in the removal and therefore loss of large amounts of alcohol with the distilled water. The loss is undesirable both because of its environmental impact and because of the added cost of replenishing the lost alcohol. In addition, the drying of the entire alcohol component of the reaction is energy and equipment intensive, especially when carried out on an industrial scale.

In view of the foregoing, a need continues to exist for new cost effective processes that provide monofunctional polyalkylene glycols containing low amounts of diol contaminants.

### BRIEF SUMMARY OF THE INVENTION

In a first aspect, the invention provides a process for making a low diol content monofunctional polyalkylene glycol of the formula (I):

RO[(CHR¹)ₕ—O]ₖ—[(CHR¹)ₘ—O]ₙ—Y (I)

wherein R is C₁-C₈ alkyl or aryl (e.g., phenyl); R¹ at each occurrence is independently H or C₁-C₈ alkyl; h and m are independently 2-6; k and n are independently zero or the average number of moles of the —[(CHR¹)ₕ—O] and [(CHR¹)ₘ—O] groups respectively, provided that k and n are not simultaneously zero; and Y is H or an alkaline metal. The process comprises:
(a) providing a first initiator comprising an alkoxide of a first alcohol;
(b) drying the first initiator to remove water;
(c) mixing a second initiator with the first initiator, wherein the second initiator comprises a second alcohol, and wherein the first alcohol and the second alcohol are the same or different and are independently selected from a compound of formula (II):

R[O(CHR²)ₚ]_{q}OH (II)
wherein R is C₁-C₈ alkyl or aryl, R² at each occurrence is independently H or C₁-C₈ alkyl; p is 2-6; and q is 0-20; and
(d) contacting the first initiator and the second initiator with one or more alkylene oxide compounds so that the alkylene oxide compounds react therein to form the monofunctional polyalkylene glycol compound of formula (I).

In a second aspect, the invention provides a composition comprising a first monofunctional polyalkylene glycol and a second monofunctional polyalkylene glycol, wherein the first monofunctional polyalkylene glycol and the second monofunctional polyalkylene glycol are independently selected from a compound of the formula (I):

RO[(CHR¹)ₕ—O]ₖ—[(CHR¹)ₘ—O]ₙ—Y (I)

wherein R is C₁-C₈ alkyl or aryl; R¹ at each occurrence is independently H or C₁-C₈ alkyl; h and m are independently 2-6; k and n are independently zero or the average number of moles of the —[(CHR¹)ₕ—O] and [(CHR¹)ₘ—O] groups respectively, provided that k and n are not simultaneously zero; and Y is H or an alkaline metal, and wherein the R group of the first monofunctionalized polyalkylene glycol is different from the R group of the second polyalkylene glycol.

### DETAILED DESCRIPTION OF THE INVENTION

The invention provides, in a first aspect, a process for making a low diol content monofunctionalized polyalkylene glycol of the formula (I). Low diol content is achieved in the invention by utilizing a dual initiator approach to the polymerization. According to this approach, it is only necessary to dry a portion of the initiator prior to proceeding to polymerization, rather than drying the entire initiator component, as in the prior art. As noted earlier, water is the primary source of diol contaminants in monofunctional polyalkylene glycol products, and reducing its presence is therefore necessary for the manufacture of low diol material.

The dual initiator approach of the invention yields several advantages over previously known systems. For instance, because the invention dries a portion of the initiator, there is reduced overall initiator loss into the surroundings. Consequently, the environmental impact of the production process is significantly mitigated. In addition, raw material costs are reduced. The process also provides enhanced flexibility in raw material use, for instance, by allowing the selection of different alcohols for the first and second initiators. As an added benefit of optionally using different alcohols for the initiator, the first initiator can be chosen, based on its boiling point, such that the drying step can be conducted at higher temperature and lower pressure, thus reducing the drying cycle time. A further advantage is that the invention is amenable to the use of conventional catalysts, such as potassium hydroxide and sodium hydroxide.

The monofunctional polyalkylene glycols of the invention are particularly suitable for use in applications where the presence of significant amounts of diols (difunctional glycols) is undesirable because of the crosslinking that the diols may cause. Such applications include, for example, the manufacture of pharmaceutical products, cement applications, and polyurethane and polyurethane prepolymer production.

The monofunctional polyalkylene glycols prepared according to the process of the invention are generally of the formula (I):

RO[(CHR¹)ₕ—O]ₖ—[(CHR¹)ₘ—O]ₙ—Y (I)

wherein R is C₁-C₈ alkyl or aryl (preferably C₁-C₈ alkyl); R¹ at each occurrence is independently H or C₁-C₈ alkyl (preferably H at each occurrence); h and m are independently 2-6; k and n are independently zero or the average number of moles of the [(CHR¹)ₕ—O] and [(CHR¹)ₘ—O] groups respectively, provided that k and n are not simultaneously zero; and Y is H or an alkaline metal.

In one particularly preferred embodiment of the invention, k in the polymer of formula (I) is zero (i.e., the [(CHR¹)ₕ—O] group is absent). Therefore, a preferred monofunctional polyalkylene glycol is of the formula (IA):

RO[(CHR¹)ₘ—O]ₙ—Y (IA)

wherein R is C₁-C₈ alkyl or aryl (preferably C₁-C₈ alkyl); R¹ at each occurrence is independently H or C₁-C₈ alkyl (preferably H at each occurrence); Y is H or an alkaline metal; m is 2-6 (preferably 2-4, more preferably 2); and n is the average number of moles of the [(CHR¹)ₘ—O] group (preferably in the range of 7 to 120).

The process for preparing low diol content monofunctional polyalkylene glycols of formula (I) comprises:
(a) providing a first initiator comprising an alkoxide of a first alcohol;
(b) drying the first initiator to remove water;
(c) mixing a second initiator with the first initiator, wherein the second initiator comprises a second alcohol, and wherein the first alcohol and the second alcohol are the same or different and are independently selected from a compound of formula (II):

R[O(CHR²)ₚ]_{q}OH (II)
wherein R is C₁-C₈ alkyl or aryl, R² at each occurrence is independently H or C₁-C₈ alkyl; p is 2-6; and q is 0-20 and
(d) contacting the first initiator and the second initiator with one or more alkylene oxide compounds so that the alkylene oxide compounds react therein to form the monofunctional polyalkylene glycol of formula (I).

Step (a) of the process is the provision of a first initiator comprising an alkoxide of a first alcohol. The alkoxide can be formed by techniques well known to those skilled in the art. Typically, the first alcohol is contacted with a catalyst under conditions suitable for alkoxide formation. Various catalysts may be used, although preferred catalysts are aqueous potassium hydroxide and aqueous sodium hydroxide. Aqueous potassium hydroxide is particularly preferred.

In a typical alkoxylation procedure, the catalyst is added to the alcohol in a solvent, such as water or methanol. Generally, about 0.01-0.5 weight percent of catalyst, based on total weight of first initiator, is used. Preferably, the weight of catalyst is 0.1-0.2 percent based on total initiator content.

Step (b) of the process of the invention is the drying of the first initiator to remove water. Drying can be conducted by a variety of methods. For instance, the first initiator may be heated to above the boiling point of water (e.g., to about 110° C.) and/or sparged with a dry inert gas, such as nitrogen. Water can also be removed by vacuum distillation at elevated temperature and/or reduced pressure (the specific temperature and pressure will depend on the alcohol being used and can be readily determined by a person of ordinary skill in the art).

It is not necessary that all traces of water be removed from the first initiator. However, in preferred embodiments, the drying of the first initiator results in a water content of 1200 ppm or less, more preferably 800 ppm or less, and even more preferably 500 ppm or less, and further preferably 300 ppm or less.

In step (c) of the process, a second initiator is mixed with the first initiator. The second initiator is a second alcohol that, in the invention, does not need to be dried in the same manner as the first alcohol. In order to minimize diol formation, however, it is preferred that the second initiator preferably be of a grade that has a water content of 1000 ppm or less, more preferably 700 ppm or less, and even more preferably 500 ppm or less. In some embodiments, the mixture of first and second initiators preferably has a total water content of 1000 ppm or less, more preferably 500 ppm or less.

In the invention, the first and second alcohols are selected so as to provide the desired terminal group or mixture of terminal groups in the monofunctional polyalkylene glycol product. In particular, the first and second alcohols are independently selected from compounds of the formula (II):

R[O(CHR²)ₚ]_{q}OH (II)

wherein R, R², p and q are as defined above.

R in formula (II) is preferably C₁-C₄ alkyl. R² at each occurrence is preferably H. In some embodiments, p is preferably 2-4, more preferably 2 or 3. In some embodiments, q is preferably 1-10, more preferably 1-5. Specific non-limiting examples of preferred alcohols include: alkanols, such as butanol, 2-methylbutanol, pentanol, 4-methyl-2-pentanol, and hexanol; ethylene glycol monoalkyl ethers such as ethylene glycol monobutyl ether, ethylene glycol mono-n-propyl ether, ethylene glycol monohexyl ether (available from The Dow Chemical Company as Butyl CELLOSOLVE™, Propyl CELLOSOLVE™, and Hexyl CELLOSOLVE™, respectively); diethylene glycol monoalkyl ethers such as diethylene glycol monomethyl ether, diethylene glycol monoethyl ether, diethylene glycol monobutyl ether, and diethylene glycol monohexyl ether (available from The Dow Chemical Company as Methyl CARBITOL™, CARBITOL™, Butyl CARBITOL™, and Hexyl CARBITOL™, respectively); alkoxytriglycols (triethylene glycol monoalkyl ethers) such as methoxytriglycol (MTG), ethoxytriglycol, and butoxytriglycol (available from The Dow Chemical Company); mono-, di-, or tri-propylene glycol alkyl ethers, including those available from The Dow Chemical Company, such as propylene glycol methyl ether (DOWANOL PM), propylene glycol n-propyl ether (DOWANOL PnP), propylene glycol n-butyl ether (DOWANO PnB), dipropylene glycol methyl ether (DOWANOL DPM), dipropylene glycol n-propyl ether (DOWANOL DPnP), dipropylene glycol n-butyl ether (DOWANOL DPnB), tripropylene glycol methyl ether (DOWANOL TPM), tripropylene glycol n-butyl ether (DOWANOL TPnB); and phenyl capped glycol ethers such as ethylene glycol phenyl ether and propylene glycol phenyl ether (DOWANOL Eph and DOWANOL PPh from The Dow Chemical Company).

The ratio of first initiator to second initiator in the process of the invention generally depends on the desired molecular weight of the final monofunctional polyalkylene glycol product. Typically, the ratio is between about 5:1 and about 1:20 by weight of first initiator to second initiator. For preparing lower molecular weight monofunctional polyalkylene glycols (e.g., MPEG of about 350 molecular weight), the weight ratio of first initiator to second initiator is preferably between about 1:10 and 1:15. For higher molecular weight material (e.g., MPEG of about 2000 molecular weight), the weight ratio is preferably between about 1:1 and 1:5.

As can be seen from the various preferred ratios of first initiator to second initiator, the catalyzed (first) initiator, which is subjected to drying, can comprise only a relatively small portion of the entire initiator content of the polymerization reaction. As a consequence, the drying is more efficiently conducted than previously known processes, in terms of the amount of initiator lost overhead during drying and the water level that can be achieved in a relatively short amount of time. Advantageously, the invention process not only dries the first initiator to a low level water content, but also dilutes this low water content by adding a second charge that has not come into direct contact with the catalyst (the primary water source).

In step (d) of the processes of the invention the first and second initiators are contacted with one or more alkylene oxide compounds under polymerization conditions. The alkylene oxides are independently selected to provide the desired formula (I). Suitable alkylene oxides contain between 2 and 6 ring carbon atoms, and may be optionally substituted, such as with an alkyl. Preferred alkylene oxides include ethylene oxide, propylene oxide, and butylene oxide, with ethylene oxide being especially preferred, particularly for the preparation of low diol content formula (IA) polymers. Preferably, the alkylene oxide is of a low moisture grade or is pre-dried to reduce water content. For instance, commercially available ethylene oxide having a water content of less than 5 ppm by weight is preferred.

The polymerization reaction is carried out in a reactor. In order to further minimize water contamination, any nitrogen directed to the reactor is preferably dried using, for example, a Drierite gas drying system. Additions to the reactor should be made in a manner that excludes contamination by atmospheric moisture. The reactor system is advantageously further dried by carrying out a reaction and discarding the first batch. The temperature during step (d) is preferably in the range of from about 80 to about 140° C., and preferably from about 110 to about 130° C.

Reactor pressure is chosen to suit the pressure rating of the reactor, and can be readily determined by a person of ordinary skill in the art. By way of example, in the synthesis of MPEG having targeted number average molecular weight of about 350, it is preferable in some embodiments that the initial, peak, and post digest pressures be in the range of 30-35 psia, 105-115 psia, and 30-35 psia, respectively. For MPEG of targeted molecular weight of 550, the preferred initial, peak, and post-digest pressures in some embodiments are 17-22, 60-70, and 35-40 psia. For MPEG of 1000 molecular weight, preferred pressures in some embodiments are 35-40, 115-125, and 77-82 psia. For MPEG of about 2000 molecular weight, preferred pressures in some embodiments are 37-43, 115-125, and 82-87 psia.

The ratio of the one or more alkylene oxides to the initiator is chosen to produce a polymer of the desired molecular weight. Typically, between about 50-95 percent oxide, based on the total weight of the production batch, is used.

Following polymerization, an acid such as acetic acid or phosphoric acid is preferably added to neutralize residual catalyst. The resulting salts may be filtered from the product or left in the product in the case of soluble salts obtained when an organic acid is employed.

The process of the invention provides monofunctional polyalkylene glycols having a low diol content. Preferably, the diol content of the product is 3 weight percent or less, more preferably 2 weight percent or less, and even more preferably 1 weight percent or less.

In a first preferred embodiment of the process of the invention, the same alcohol of formula (I) is used for both the first alcohol (which is alkoxylated to form the first initiator) and the second alcohol/initiator. Any alcohol of formula (II), including those specifically recited above, may be used for this embodiment. Particularly preferred alcohols are methoxytriglycol (MTG) and Methyl Carbitol.

This first embodiment yields a monofunctional polyalkylene glycol product in which the inert terminal group is the same throughout the product. In this embodiment, the capping group (R in formula (I)) is preferably C1-C3 alkyl, more preferably methyl. Further, R¹ in formula (I) is preferably hydrogen at each occurrence.

One of the main advantages of this first embodiment, and the process of the invention as a whole, over prior art systems is that only the portion of the total initiator that is subjected to alkoxylation catalyst (and its resultant addition and generation of water) is dried. Consequently, loss of initiator to the surroundings is significantly reduced, mitigating the environmental and cost impacts of the process. Further, the invention process reduces the need for epoxide adjustment to account for the lost initiator, particularly in the case of higher vapor pressure initiators such as Methyl Carbitol, and therefore provides a more predictable molecular weight of the final product.

In a second preferred embodiment of the process of the invention, the first alcohol is a different compound from the second alcohol, but the terminal group in each alcohol (R in formula (II)) is the same. Any alcohol of formula (II), including those specifically recited above, may be used for this embodiment. Particularly preferred first and second alcohol combinations include: methyoxytriglycol (MTG)/Methyl Carbitol and methoxytetraglycol/Methyl Carbitol.

The process of the invention, as illustrated by this second embodiment, in allowing the selection of different alcohols for the first and second initiator, provides several significant advantages over the prior art. For example, since the first initiator is subjected to drying at increased temperature and/or reduced pressure, in some embodiments it is preferred that a lower vapor pressure (higher boiling point) material be used. By using a low vapor pressure material, loss of alcohol during drying can be reduced even further. Because it is not necessary to subject the second alcohol to drying, there is no particular need to select a low vapor pressure material for the second alcohol. Illustrative of this advantage is the MTG/Methyl Carbitol combination (as first and second alcohols, respectively) described in the Examples below.

The MTG/Methyl Carbitol combination further demonstrates the flexibility provided by the ability to use different alcohols for the initiator. In particular, the combination results in a product with lower diol content than using only MTG as the first and second alcohols. This is because the amount of diethylene glycol (DEG) (a contaminant with crosslinking potential) typically present in commercial MTG is over 700 ppm. Because of the closeness of the boiling point of DEG (bp 245° C.) to that of MTG, DEG cannot be easily removed from MTG by distillation. On the other hand, the diol content (primarily ethylene glycol) of Methyl Carbitol is only about 200 ppm on average and water content is typically less than 100 ppm. Therefore, the MTG/Methyl Carbitol combination results in lower diol content in the product than using MTG alone (see Examples below).

A further advantage of the second embodiment is that it allows flexibility in initiator selection. The MTG/Methyl Carbitol combination provides one example of this advantage. Since MTG supply is extremely tight world-wide because of MTG's usage in brake fluids and in gas treating applications, it is desirable to use the more abundant Methyl Carbitol. The mixed feed approach allows the reduction of MTG consumption by over 50%.

In a third preferred embodiment of the process of the invention, the terminal R group of the first alcohol is different from the terminal R group in the second alcohol. This embodiment provides monofunctional polyalkylene glycols having mixed terminal capping groups. Any combination of alcohols of formula (II), including those specifically recited above, may be used for this embodiment. A preferred combination is Methyl Carbitol as the first alcohol and butanol as the second alcohol. Other preferred first alcohol/second alcohol combinations include: MTG/ethanol, MTG/methanol, and MTG/Butyl Carbitol.

In its second aspect, the invention provides a composition comprising a mixture of a first monofunctional polyalkylene glycol and a second monofunctional polyalkylene glycol, wherein the first monofunctional polyalkylene glycol and the second monofunctional polyalkylene glycol are independently selected from a compound of the formula (I):

RO[(CHR¹)ₕ—O]ₖ—[(CHR¹)ₘ—O]ₙ—Y (I)

wherein R is C₁-C₈ alkyl or aryl; R¹ at each occurrence is independently H or C₁-C₈ alkyl; h and m are independently 2-6; k and n are independently zero or the average number of moles of the —[(CHR¹)ₕ—O] and [(CHR¹)ₘ—O] groups respectively, provided that k and n are not simultaneously zero; and Y is H or an alkaline metal, and wherein the R group of the first monofunctional polyalkylene glycol is different from the R group of the second monofunctional polyalkylene glycol.

In this aspect of the invention, R¹ at each occurrence is preferably H. R is preferably C₁-C₈ alkyl, more preferably methyl, ethyl, or butyl (provided that it differs between the first and second monofunctional polyalkylene glycols). Further preferably, k is zero and m is 2.

In a particularly preferred embodiment of the second aspect of the invention, the first and second monofunctional polyalkylene glycols are independently selected from a compound of the formula (IA):

RO[(CHR¹)ₘ—O]ₙ—Y (IA)

wherein R, m, n, and Y are as defined above, and wherein the R group of the first monofunctional polyalkylene glycol is different from the R group of the second polyalkylene glycol.

In a further preferred embodiment, the mole ratio of first monofunctional polyalkylene glycol to second monofunctional polyalkylene glycol in the composition is between about 99:1 and 1:99, more preferably between about 90:10 and 10:90.

As demonstrated by the Examples below, the mixed polyalkylene glycols of the invention are functionally equivalent to uniformly monofunctional polyalkylene glycols, such as MPEGs and therefore may be used in the same applications as MPEGs. One advantage of this aspect of the invention is that it permits use of raw materials that may be more abundant or less expensive to yield glycols with substantially analogous properties to uniformly terminated glycols.

The monofunctional polyalkylene glycol compounds prepared as described above are of the formula (I): RO[(CHR¹)ₕ—O]ₖ—[(CHR¹)ₘ—O]ₙ—Y. It should be understood that this is an empirical formula, and that Y can be a mixture of hydrogen and an alkali metal and that when Y is an alkali metal the compound may be dissociated. Further, while for convenience the repeat units of the polymers are as shown, it should also be noted that when both k and n units are present, the polymers are not necessarily block copolymers. Rather, the invention encompasses all possible distributions of the k and n units in the polymers, including randomly distributed k and n units, alternately distributed k and n units, as well as partially and fully block or segmented copolymers.

Although there is no particular limitation on the molecular weight of polymers prepared by any of the processes of the invention, in some embodiments the polymers preferably have a number average molecular weight of between about 300 and about 5500. Polymers with average molecular weights of about 2000, about 1000, about 750, or about 600 are particularly preferred.

“Alkyl,” as used in this specification, encompasses straight and branched chain aliphatic groups having from 1-8 carbon atoms, more preferably 1-6 carbon atoms. Preferred alkyl groups include, without limitation, methyl, ethyl, propyl, isopropyl, butyl, isobutyl, sec-butyl, tert-butyl, pentyl, and hexyl. Particularly preferred are methyl, ethyl, and propyl.

An “aryl” group is a C6-C12 aromatic moiety comprising one to three aromatic rings. Preferably, the aryl group is a C6-C10 aryl group. A preferred aryl group is phenyl.

The following examples are illustrative of the invention but are not intended to limit its scope.

### Examples

### Example 1

### Production of Low Diol MPEG 420

Approximately 132.2 lbs of 45% aqueous KOH catalyst are added to 3000 lbs of Silicon Grade methoxytriglycol (MTG). The catalyzed initiator is then dried at 125° C. at approximately 30 mmHg until a 0.03 weight percent water content is reached. Approximately 37,776 lbs of MTG (second initiator) are mixed with the dried first initiator, and the mixture heated to 130° C. and then transferred to a reactor for ethylene oxide (EO) feed. The EO (63,968 lbs) is fed to the initiator mixture at 130° C. and digested for 87 min at this temperature. Following reaction, the product is neutralized to pH 4.5-7.5 with phosphoric acid, and the resulting salts allowed to crystallize by cooling to 80° C. for 60 minutes, and filtering. Diol content in the product is measured using HPLC. Table 1 shows the diol level for various batches of low diol MPEG made with the procedure of the invention. As can be seen, all the MPEGs contain advantageously low levels of diol.

TABLE 1
Diol Levels for Low Diol MPEGs produced by the procedure
of Example 1.

| | | Weight % | Mole % | Diol Molecular |
|---|---|---|---|---|
| Run | Targeted Mn | Diol | Diol | Weight |
| 1 | 350 | 0.42 | 0.3 | 550 |
| 2 | 350 | 0.30 | 0.2 | 580 |
| 3 | 350 | 0.26 | 0.2 | 520 |
| 4 | 420 | 0.29 | 0.19 | 620 |

Table 2 shows comparative data on diol levels for an MPEG 420 produced using solid KOH catalyst and then drying the catalyzed initiator by sparging with nitrogen (entries 5 and 6, which are non-invention examples). Entry 4 is prepared according to the procedures of the invention.

TABLE 2
Diol Level comparison between MPEG 420 produced
by the above method and a drying method
that does not employ the partial charge step described above.

| | | Weight % | Mole % | Diol Molecular |
|---|---|---|---|---|
| Entry | | Diol | Diol | Weight |
| 4 | MPEG 420 (invention) | 0.29 | 0.19 | 620 |
| 5 | MPEG 420 | 0.85 | 0.89 | 400* |
| | (comparative) | | | |
| 6 | MPEG 420 | 0.27 | 0.35 | 320* |
| | (comparative) | | | |

### Example 2

### Production of Low Diol MPEG 1000 with MTG Initiator

In a jacketed 5-gallon reactor, approximately 10.2 grams of aqueous KOH catalyst are added to 840 g of MTG. This catalyzed initiator is then dried to approximately 205 ppm water by sparging with nitrogen at 110° C. Approximately 832 grams of the dry catalyzed initiator are then mixed with approximately 3789 grams of methyl carbitol. Approximately 14200 grams of ethylene oxide are fed semi-continuously to an initiator at 130° C. over a period of several hours after which time the product is allowed to cool and approximately 4.1 grams of 45% aqueous phosphoric acid are added to neutralize the product and stop the reaction. The resulting product is analyzed for molecular weight and molecular weight distribution by GPC and for diol content by HPLC. See Table 3.

### Example 3

### Production of Low Diol MPEG 1000 with MTG/Methyl Carbitol Initiator

An analogous procedure to Example 2 is used to prepare MPEG 1000, except that MTG is used as the first initiator and Methyl Carbitol as the second initiator.

Table 3 shows comparisons in initiator loss during drying for Examples 2 and 3.

TABLE 3
Amount of initiator lost during drying as a function of initiator.

| | | | Water | Initiator |
|---|---|---|---|---|
| | | | Content | Loss |
| | | | After | During |
| Entry | Sample Description | Initiator | Drying | Drying |
| 7 | MPEG 1000 Polyglycol | Methyl Carbitol | 285 ppm | 38.5% |
| 8 | MPEG 1000 Polyglycol | Methyl Carbitol | 107 ppm | 17.7% |
| 9 | MPEG 1000 | Methyl Carbitol | 275 ppm | 22.3% |
| 10 | MPEG 1000 | MTG/ | 205 ppm | 9.4% |
| | | Methyl Carbitol | | |

### Example 4

### Comparison Between MPEG 1000 of the Invention with Prior Art Material

Table 4 below shows a comparison of three different batches of MPEG 1000 prepared by the process of the invention (entries 11, 12, and 13) and two comparative examples from the prior art process of WO2006/061110 A1 (entries 14 and 15). From the data in the table, it is shown that the MPEG 1000 produced with the mixed feed approach is “low” in diol content and functionally identical to the MPEGs produced with MTG or methyl carbitol alone as noted by the molecular weight distribution (Mw/Mn) as determined by gel permeation chromatography. The diol content in Entry 13 is higher than the diol content in Entries 11 and 12 likely because of increased diol level in the initiator and water level in the EO in Entry 13 compared to Entries 11 and 12.

TABLE 4
Comparison of diol content and molecular weight distribution
of samples produced with different initiators.

| | | | | Diol |
|---|---|---|---|---|
| | | | | Content |
| Entry | Sample Description | Initiator | Mw/Mn | (wt %) |
| 11 | MPEG 1000 Polyglycol | Methyl Carbitol | 1.05 | 0.70 |
| 12 | MPEG 1000 Polyglycol | MTG | 1.05 | 0.70 |
| 13 | MPEG 1000 Polyglycol | Methyl Carbitol/ | 1.04 | 1.56 |
| | | MTG | | |
| 14 | MPEG 3000 Comparison | Methyl Carbitol | Not | ~2.6 |
| | Example 1 from | | Reported | |
| | WO2006/061110 A1 | | | |
| 15 | MPEG 4000 Comparison | Methanol | Not | ~26 |
| | Example 1 from | | Reported | |
| | WO2006/061110 A1 | | | |

### Example 5

### Preparation of MPEG/BPEG 1000 Blend

This Example demonstrates the preparation of a mixed cap polyglycol product from a mixed initiator of methyl carbitol and 1-butanol. The product is a low impurity monol (monofunctional) material suitable as a replacement to MPEGs, particularly low diol MPEGs, such as for the applications discussed above.

In a jacketed 5-gallon reactor, 3.5 grams of solid KOH catalyst is added to 350 g of methyl carbitol at 80° C. This catalyzed initiator is then dried to approximately 140 ppm water by sparging with nitrogen. 272 grams of the dry catalyzed initiator is then mixed with approximately 1238.7 grams of 1-butanol. The initiator mixture is sampled for water content. The total mixed initiator weight after sampling is approximately 1487.5 grams. Approximately 17225 grams of ethylene oxide are added to the initiator at 130° C. The reaction is carried out for several hours after which time the product is allowed to cool and 5.19 grams of 45% aqueous phosphoric acid are added to neutralize the product and stop the reaction. The resulting product is analyzed for molecular weight and molecular weight distribution by GPC and for diol content by HPLC. Viscosity and melting point of the resulting sample are also measured and these properties are compared to a conventional MPEG 1000 sample as shown in Table 5.

TABLE 5
Comparison of physical properties for MPEG 1000 made with
MTG to monofunctional PAG made with a mixed initiator feed.

| Sample | | | Melt | Diol | Viscosity | Viscosity | Viscosity |
|---|---|---|---|---|---|---|---|
| Description | Initiator | Mw/Mn | Point | Content (wt %) | at 40° C. | at 60° C. | at 100° C. |
| MPEG 1000 | MTG | 1.05 | 38 | 0.70 | 69.3 | 31.9 | 10.7 |
| Polyglycol | | | | | | | |
| MPEG/BPEG 1000 | Methyl Carbitol/ | 1.06 | 37 | 0.91 | 69.1 | 33.9 | 10.4 |
| Polyglycol | Butanol | | | | | | |

The properties of the mixed initiator sample are almost identical to the MPEG sample as is their appearance. The viscosities are also very similar over a range of temperatures. This example demonstrates that such a mixed feed approach can be used to produces samples that are functionally equivalent to similar molecular weight MPEGs typically used in various applications.

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A process for making a monofunctional polyalkylene glycol of the formula (I):
RO[(CHR¹)ₕ—O]ₖ—[(CHR¹)ₘ—O]ₙ—Y (I)
wherein R is C₁-C₈ alkyl or aryl; R¹ at each occurrence is independently H or C₁-C₈ alkyl; h and m are independently 2-6; k and n are independently zero or the average number of moles of the [(CHR¹)ₕ—O] and [(CHR¹)ₘ—O] groups respectively, provided that k and n are not simultaneously zero; and Y is H or an alkaline metal, the process comprising:
(a) providing a first initiator comprising an alkoxide of a first alcohol;
(b) drying the first initiator to remove water;
(c) mixing a second initiator with the first initiator, wherein the second initiator comprises a second alcohol, and wherein the first alcohol and the second alcohol are the same or different and are independently selected from a compound of formula (II):
R[O(CHR²)ₚ]_{q}OH (II)
wherein R is C₁-C₈ alkyl or aryl, and R² at each occurrence is independently H or C₁-C₈ alkyl, p is 2-6, and q is 0-20; and
(d) contacting the first initiator and the second initiator with one or more alkylene oxide compounds so that the alkylene oxide compounds react therein to form the monofunctional polyalkylene glycol of formula (I).

2. A process according to claim 1 wherein the monofunctional polyalkylene glycol is of the formula (IA):
RO[(CHR¹)ₘ—O]ₙ—Y (IA)
wherein R is C₁-C₈ alkyl or aryl; R¹ at each occurrence is independently H or C₁-C₈ alkyl; m is 2-6; n is the average number of moles of the [(CHR¹)ₘ—O] group; and Y is H or an alkaline metal.

3. A process according to claim 1 wherein the first alcohol and the second alcohol are different compounds.

4. A process according to claim 1 wherein the alkoxide is prepared by reacting the first alcohol with aqueous potassium hydroxide.

5. A process according to claim 1 wherein the first alcohol has a boiling point greater than the boiling point of water.

6. A process according to claim 1 wherein the first initiator has a water content of 1200 ppm or less following the drying of step (b).

7. A process according to claim 1 wherein the second initiator has a water content of 1000 ppm or less.

8. A process according to claim 1 wherein the mixture of step (c) has a total water content of 500 ppm or less.

9. A process according to claim 1 wherein the ratio of the first alcohol to the second alcohol by weight ranges from about 5:1 to about 1:20.

10. A process according to claim 1 wherein the first and second alcohols are independently selected from: alkanols; ethylene glycol monoalkyl ethers; diethylene glycol monoalkyl ethers; triethylene glycol monoalkyl ethers; mono-, di-, or tripropylene glycol alkyl ethers; and phenyl capped glycol ethers.

11. A process according to claim 1 wherein the first alcohol is a triethylene glycol monoalkyl ether and the second alcohol is a diethylene glycol monoalkyl ether.

12. A process according to claim 1 wherein the first alcohol is methoxytriglycol and the second alcohol is diethylene glycol monomethyl ether.

13. A process according to claim 1 wherein the first alcohol is a triethylene glycol monoalkyl ether and the second alcohol is an alkanol.

14. A process according to claim 1 wherein the first alcohol is methoxytriglycol and the second alcohol is butanol.

15. A process according to claim 1 wherein the first alcohol and the second alcohol are both methoxytriglycol.

16. A process according to claim 1 wherein the first alcohol and the second alcohol are the same.

17. A composition comprising a mixture of a first monofunctional polyalkylene glycol and a second monofunctional polyalkylene glycol, wherein the first monofunctional polyalkylene glycol and the second monofunctional polyalkylene glycol are independently selected from a compound of the formula (I):
RO[(CHR¹)ₕ—O]ₖ—[(CHR¹)ₘ—O]ₙ—Y (I)
wherein R is C₁-C₈ alkyl or aryl; R¹ at each occurrence is independently H or C₁-C₈ alkyl; h and m are independently 2-6; k and n are independently zero or the average number of moles of the [(CHR¹)ₕ—O] and [(CHR¹)ₘ—O] groups respectively, provided that k and n are not simultaneously zero; and Y is H or an alkaline metal, and wherein the R group of the first monofunctional polyalkylene glycol is different from the R group of the second polyalkylene glycol.

18. A composition according to claim 17 wherein the first monofunctional polyalkylene glycol and the second monofunctional polyalkylene glycol are independently selected from a compound of the formula (IA):
RO[(CHR¹)ₘ—O]ₙ—Y (IA)
wherein R is C₁-C₈ alkyl or aryl; R¹ at each occurrence is independently H or C₁-C₈ alkyl; m is 2-6; n is the average number of moles of the [(CHR¹)ₘ—O] group; and Y is H or an alkaline metal, and wherein the R group of the first monofunctional polyalkylene glycol is different from the R group of the second polyalkylene glycol.
